(19) United States
(12) Patent Application Publication
(10) Pub. No.: US 2009/0204088 A1
(43) Pub. Date: Aug. 13, 2009

(54) EASILY REMOVABLE UNDERWEAR

(76) Inventors: STACY LYNNE STEARMAN, Rockville, MD (US); Eric Haynes Stearman, Rockville, MD (US)

Correspondence Address: WILLIAM D. HARE, 66 WITHERSPOON STREET, STE. 1, PMB 317, PRINCETON, NJ, 08542-9944, US

(21) Appl. No.: 12/350,760

(22) Filed: Jan. 8, 2009

Related U.S. Application Data
(60) Provisional application No. 61/020,384, filed on Jan. 10, 2008.

(51) Int. Cl. A61F 13/56

(52) U.S. Cl. 604/385.01; 604/386

(57) ABSTRACT

The invention generally relates to easily removable fabric undergarments used in toilet training children. The fabric undergarment includes a front panel and a rear panel. The front panel includes an upper edge and two side panels extending from opposite sides of the front panel, each side panel terminating in a side edge that includes a first connecting means. The rear panel includes an upper edge and two side panels extending from opposite sides of the front panel, each side panel terminating in a side edge that includes a second connecting means. Each of the first connecting means is configured to mate with the opposite second connecting means to form a closed waist loop for encircling the waist of the child wearing the undergarment formed by the front panel, the rear panel, and the side panels, and a pair of closed leg loops for encircling the legs of the child wearing the undergarment. The leg loops and waist loop each being configured to have a repeatable and consistent diameter.

### CROSS-REFERENCE TO RELATED APPLICATIONS

This application claims priority from U.S. provisional patent application No. 61/020,384, filed on Jan. 10, 2008, the contents of which are incorporated herein in their entirety by reference.

### TECHNICAL FIELD

The field of the invention generally relates to easily removable fabric undergarments used in toilet training children.

### BACKGROUND

When toddlers and young children begin toilet training, one often sees a progression of types of undergarments the child wears based on the level of control they have over urination and excretion of fecal matter. They begin with bulky, extremely absorbent diapers with the absorbent material throughout the crotch portion of the diaper, walls and in particular the portion of the diaper covering the buttocks. These diapers fit snug around the legs to prevent excrement from passing through the diaper and down the legs onto clothing. As such, the diapers are bunched up around the legs to hold in excrement. When the child soils him or herself, the parent must remove the soiled diaper to clean the child's genitals and buttocks and then put a new, clean diaper on the child. As the child gains more control over urinary and other bodily functions, they graduate to using pull-on or pull-up diapers that are less bulky and more like traditional underwear. These pull-on and pull-up diapers let the child pull off or pull on their own diapers so that they can begin using toilet facilities for urinating and excreting fecal matter. Like diapers, these typically fit snug around the legs to contain the excrement and also are made of absorbent materials to absorb liquids and excrements. Again, these pull-on or pull-up diapers are bunched around the legs.

Currently, in the last stage of toilet training the child uses training pants. At this stage, the child has fairly good control over urination and bodily functions, but occasionally wets or soils his or her pants with urine and fecal matter. These pants are made of cotton typically, but are thicker to better absorb urine. Training pants also have tighter leg bands to prevent leakage of urine and fecal matter. Again, the training pants, like diapers, are bunched up around the legs. Some training pants have a removable absorbent insert that can be changed out when the child urinates in their pants. When soiled or needing removal, training pants typically must be pulled down the child's legs and over the ends of the feet, requiring removal of shoes, socks, tights, pants, and any other articles of clothing on the lower half of the child's body. Replacement of the training pant similarly requires pulling the pant back over the child's feet and up the legs. Some training pants use a hook and pile connecting means to connect the front and back panels of the underwear along the side edges. These connecting means are often bulky and tend to be scratchy against the child's skin.

When the child has learned to control his or her bodily functions of urination and excretion of fecal matter, the child is considered to be toilet-trained. The child begins wearing regular underwear that they pull on and take off by themselves. The underwear is not bulky because it is typically made of single layers of cotton and other material, though it may have a dual-layer crotch. Although the child is considered toilet-trained, many children occasionally still accidentally urinate or excrete fecal matter in small amounts. Often the urine or fecal matter amount is so small that the underwear contains it, and the outer garments remain unsoiled. Underwear typically is made of a material that is of minimal absorbency and is designed not to absorb fluids but cover the skin. Unlike diapers and training pants, underwear is designed for comfort and as a result the underwear fits loosely around the legs such that it fits without bunching because there is no need to contain or hold in excrement.

There are a number of patents that attempt to provide solutions for children who are undergoing toilet training or otherwise provide easily removable clothing. U.S. Pat. No. 4,581,772 to Smith, titled “Training Panty,” discloses a training panty that includes a pair of pants having a front panel and a rear panel joined at the crotch. The training panty includes releasable contact fastening members that are positioned on opposing surfaces of front panel and rear panel, respectively along the lateral edges thereof so that front panel and rear panel can be releasably secured together to define a waistband and a pair of identical leg openings. It is believed that the training panty disclosed by Smith is an absorbent material to absorb fluids from the wearer. The leg openings fit snug around the legs using a leg casing; it is believed that the snug fit is designed to prevent excrement from passing out of the training pants and down the legs.

U.S. Pat. No. 4,675,918 to O'Brien, titled “One Piece Brief with Hook and Pile Closures,” discloses a design for briefs that can be put on and removed without a person having to bend over. The brief includes an upper band portion with front and rear panels. A short tab portion descends from the center of the front panel while a longer crotch portion descends from the center of the rear panel. Fastening tapes are provided at the edges of the front and rear panels and at the lower edges of the tab and crotch portions. The front and rear panels are connected at the side and are connectable at the opposite side and crotch using the fastening tape. A resilient waist band extends along a top edge of the upper band portion and carries at one end an outwardly extending fastening tape which engages another fastening tape at an opposite end of the waist band to be used in properly aligning side edges of the front and rear panels.

U.S. Pat. No. 4,258,440 to McGowan, titled “Clothes for the Physically Handicapped,” discloses a design for a garment adapted for wear by physically handicapped persons who are incapable of executing normal dressing movements. The garment comprises a front panel applicable to the front of the handicapped person's torso and a rear panel applicable to the back of said person's torso. The front and rear panels each have opposed side edge margins and fastening means distributed thereon for simultaneously assembling and securing the garment to the wearer's torso by interconnecting the front and rear panel along their opposing edge margins in torso supported relation in the worn condition of the garment, without the necessity of normal dressing movement.

U.S. Pat. No. 6,579,273 to Dupuy, titled “Reusable baby diaper having reusable absorbent insert,” discloses a pant that has an absorbent insert and is made of multiple layers, including a waterproof outer layer. The pant is an adjustable, form fitted, reusable diaper for infants that includes a reusable or disposable pad insertable between a soft permeable inner panel and an impermeable outer panel laminated with either a soft pile or an elastomeric non-woven material. The diaper has an hourglass configuration when open and is provided with expandable leg openings and waistband and fitted with hidden snap closures arranged to provide waist adjustment independently of leg opening adjustment and vise-versa. The diaper's use of a high pile, permeable inner panel such as polar fleece wicks moisture quickly away from the infant's body. The '273 Patent states that the pad may then be removed and replaced without removal of the diaper from the infant.

U.S. Pat. No. 4,619,649 to Roberts, titled “Disposable toddler training panty,” discloses a training pant that is made of multiple layers, including a waterproof outer layer. This disposable toddler training panty has a thin plastic outer layer provided on an inner surface with a soft inner lining. The panty has an elastic waistband and legbands, a thin absorbent padded crotch area, and separable side seams from the waistband to the legband on both sides to allow for removal of the toddler panty in the event of the soiling of same.

U.S. Pat. No. 4,615,695 and U.S. Pat. No. 5,087,253, both to Cooper, are titled “Combination diaper training pant for children and adults,” and disclose training pants that are made of multiple layers, including a waterproof outer layer. The training pant may be made in a one-piece configuration, which would be disposable, or a two-piece configuration, which would be washable. An outer water-resistant portion is formed in the shape of a pair of training pants and has an inner water-absorbing portion attached or positioned on the outer portion. An inner padding is positioned between the outer water-resistant portions and the inner water-absorbing portion.

U.S. Pat. No. 2,691,376 to Tunmell, titled “Adjustable pinless diaper,” discloses an adjustable pinless diaper that provides a diaper free of pins for its use, absolutely safe, and adapted to remain in its proper position for use no matter how active the child may be. This reference is designed to be used as a diaper, and made of any materials “suitable for that purpose.” This reference also has an adjustable size waistband.

U.S. Pat. No. 6,478,786 to Glaug et al., titled “Protective underwear,” discloses a protective underwear having a crotch portion, a waist portion, a front belly portion and a rear back portion. The underwear includes an absorbent core. The underwear includes a pair of leg openings, each of which has a first arcuate section, a second arcuate section, and an intermediate section. The first arcuate section includes at least one arcuate elastic thread, as does the second arcuate section; the threads are spaced by a gap. Glaug et al. also discloses an absorbent core for collecting any fluid and an impermeable layer that contains any fluid in the absorbent core and keeps it from wetting the underwear's outside surface.

U.S. Pat. No. 7,150,731 to Cazzato et al., titled “Disposable absorbent underpants,” discloses disposable underpants that includes multiple layers, including an absorbent pad and a liquid-impermeable baffle in the crotch area. The underpants appear to fit snug around the legs.

U.S. Pat. No. 4,610,680 to LaFleur, titled “Disposable training panty,” discloses a disposable training panty that includes an outer impermeable layer, an inner absorbent layer, an enclosed waist opening and enclosed leg openings. Portions of a panty member can be separated from each other to open the periphery of waist opening of the panty member and the leg openings. The panty member includes overlapping flaps that are secured to each other by hook and pile fastening straps.

### SUMMARY

In one general aspect, a fabric undergarment for use in toilet training a child wearing the undergarment and includes a front panel and a rear panel. The front panel includes an upper edge and two side panels extending from opposite sides of the front panel, each side panel terminating in a side edge that includes a first connecting means. The rear panel includes an upper edge and two side panels extending from opposite sides of the front panel, each side panel terminating in a side edge that includes a second connecting means. Each of the first connecting means is configured to mate with the opposite second connecting means to form a closed waist loop for encircling the waist of the child wearing the undergarment formed by the front panel, the rear panel, and the side panels, and a pair of closed leg loops for encircling the legs of the child wearing the undergarment. The leg loops and waist loop each being configured to have a repeatable and consistent diameter.

Embodiments of the fabric undergarment may include one or more of the following features. For example, the side panels extending from the front panel may extend further than the side panels extending from the rear panel. The side panels extending from the rear panel may extend further than the side panels extending from the front panel. One of the side panels may extend from the front panel further than the mating panel that extends from the rear panel and the other side flap may extend further from the rear panel than the mating panel that extends from the front panel.

The connecting means may be one or more of a button or button hole or a snap and a receiving snap. The leg loops fit loosely around the legs when the child wears the undergarment. The fabric may have a minimal absorbance. The fabric may consist of a single layer or at least one layer.

The side edges may include a reinforced fabric. The side edges may include a reinforced fabric on an inner side of the side edge and an outer side of the side edge.

In another general aspect a method of changing a fabric undergarment on an individual without the need to remove outer garments from the legs of the individual includes providing a fabric undergarment and changing the undergarment. Providing the undergarment to the individual includes providing an undergarment that includes a front panel and a rear panel. The front panel has an upper edge and two side panels extending from opposite sides of the front panel, each side panel terminating in a side edge that includes a first connecting means. The rear panel has an upper edge and two side panels extending from opposite sides of the front panel, each side panel terminating in a side edge that includes a second connecting means. Each of the first connecting means is configured to mate with the opposite second connecting means to form a closed waist loop for encircling the waist of the child wearing the undergarment formed by the front panel, the rear panel, and the side panels, and a pair of closed leg loops for encircling the legs of the child wearing the undergarment. The leg loops and waist loop each being configured to have a repeatable and consistent diameter.

Removing the undergarment from the individual without removing the outer garments in their entirety from the legs of the individual wearing the outer garment includes the steps of unfastening the connecting means on the side panels and pulling the undergarment between the legs.

Embodiments of the method may include one or more of the following. For example, the method may further include placing the undergarment on the individual without removing the outer garments in their entirety from the legs of the individual wearing the outer garment by pulling the undergarment between the legs and fastening the connecting means on the side panels.

The side panels extending from the front panel may extend further than the side panels extending from the rear panel. The side panels extending from the rear panel may extend further than the side panels extending from the front panel. One of the side panels may extend from the front panel further than the mating panel that extends from the rear panel and the other side flap may extend further from the rear panel than the mating panel that extends from the front panel.

The connecting means may be one or more of a button or button hole, or a snap and a receiving snap.

The side edges may include a reinforced fabric. The side edges may include a reinforced fabric on an inner side of the side edge and an outer side of the side edge.

The leg loops may fit loosely around the legs when the child wears the undergarment. The fabric may have a minimal absorbance. The fabric may consist of a single layer or at least one layer.

The details of various embodiments of the invention are set forth in the accompanying drawings and the description below. Other features and advantages of the invention will be apparent from the description, the drawings, and the claims.

### DESCRIPTION OF THE DRAWINGS

FIG. 1 is a front perspective view of a pair of snap underwear in a wearable configuration.

FIG. 2 is a top perspective view of a pair of snap underwear in an open, interior position showing the front and rear panels.

FIGS. 3 and 4 are front and top perspective views of a pair of underwear garments that use cloth strips to connect the garment for wearing.

### DETAILED DESCRIPTION

In dealing with the toilet training process of children, the inventors recognized that there is no interim undergarment between training pants or pullups and regular underwear that allows convenient removal and replacement. This interim garment needs to allow for removal and replacement without removal of clothing articles from the lower half of the child's body, but also must be made of a thin material to fit properly under the clothes designed for children at this age. The thin material does not need to be an absorbent material, such as the material typically used in a diaper or training underwear. Instead, the material should be configured to be of a thickness and type that is similar to or the same as conventional underwear, which is not designed to absorb material such as body excrements. This is in contrast to a cloth or synthetic (e.g., acrylate) diaper, which is designed to absorb fluids, and training underwear, which is designed to absorb fluids, but not as much fluid as a diaper. Diapers typically also have a material or layer that prevents the flow of fluid out of the diaper.

This undergarment should be comfortable, having looser leg bands (e.g., no leg casings), and having no scratchy surfaces against the child's skin. The inventors also recognized that there is a need for a similar undergarment for people of limited mobility and agility, such as the elderly, hospitalized, handicapped, maternity, infirm, etc. The undergarment also can be used by people in general, not just children or those of limited mobility and agility.

The present snap underwear described herein resolves the issues of material bulkiness and inconvenience of removing outer articles of clothing to replace just slightly soiled underwear. It also provides a snap connecting means that is not scratchy against the child's or wearer's skin. The snap connecting means is also a connecting mechanism that children are familiar with and can do by themselves, making this an ideal interim garment between training pant and regular underwear. In an alternative embodiment the snaps can be replaced by or augmented with one or more of hooks and piles, buttons and button holes, buttons and loops to receive the button, slidable fastener, etc. Each of these individually has advantages to the wearer, such as ease of removal, ease of use, manufacturability, cost, etc. As such, it should be understood that the reference to the term snap also can be used to reference to other fastening means such as hooks and piles, buttons and button holes, buttons and loops to receive the button, slidable fastener, etc.

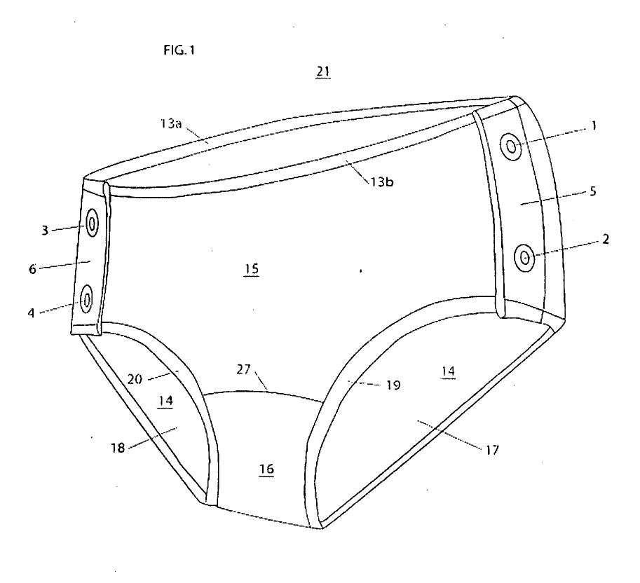

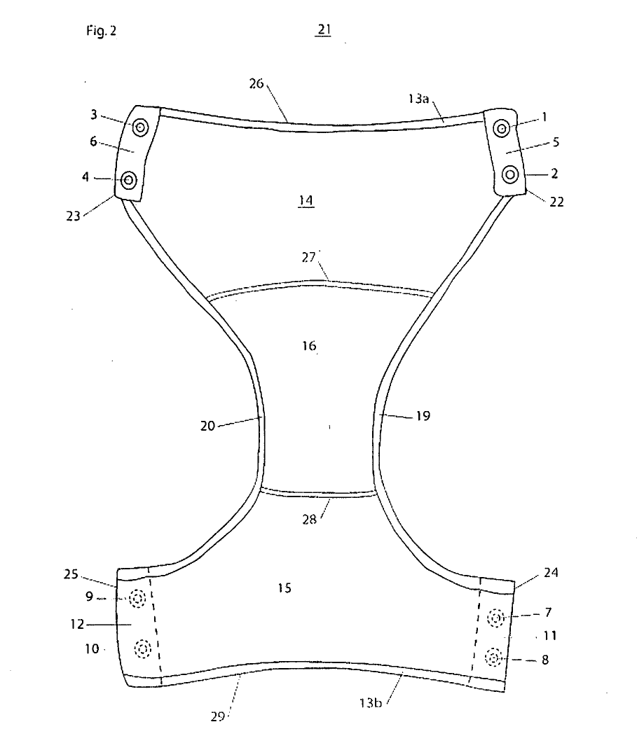

The inventor has developed a pair of snap underwear as shown by the drawings in FIGS. 1 and 2. Each drawing has reference numbers that designate like or corresponding parts in each perspective view. FIG. 1 shows a frontal perspective of the snap underwear. FIG. 2 shows an open, interior perspective of the snap underwear. As best seen in FIG. 2, the snap underwear 21 includes a front panel 15 and a rear panel 14. The underwear 21 also includes a waistband 13a and 13b, which is a conventional elastic waistband that encircles the child's waist entirely when the snap 1 is connected to receiving snap 8 and snap 3 is connected to receiving snap 10. The waistband is permanently attached to the front panel 15 and rear panel 14. The underwear 21 has two leg holes 17 and 18. Leg hole 17 is formed when snap 2 is connected to receiving snap 7, thus creating leg band 19. Leg hole 18 is formed when snap 4 is connected to receiving snap 9, thus creating leg band 20. Snaps 1 and 2 are permanently attached to reinforcing strip 5. Snaps 3 and 4 are permanently attached to reinforcing strip 6. Snaps 7 and 8 are permanently attached to reinforcing strip 11. Snaps 9 and 10 are permanently attached to reinforcing strip 12. The underwear 21 further includes the typical dual-layer crotch area 16, although a single-layer crotch area, a triple layer crotch area, or more, may be used instead. However, this potentially thicker area is limited to the crotch area.

Underwear 21 is constructed of typical undergarment materials that are flexible, such as one or more of cotton, lycra, spandex, polyester or nylon or the like, with a single-, dual-, or multi-layer crotch 16 preferably made with a cotton inner layer. The waistband formed by 13a and 13b is constructed in typical fashion by sewing elastic onto the top edge 26 of the front panel 15 and the bottom edge 29 of the rear panel 14. The leg bands 19 and 20 are most likely constructed of elastic sewn to the edges of the cloth that includes portions of the rear panel 14, front panel 15, and crotch 16, that form leg holes 17 and 18, respectively. The elastic leg bands are constructed such that the leg bands do not bunch around the legs. It is important that the undergarment have the feel of conventional underwear while yet having the ease of removal and replacement. Note that the elastic for both the waistband formed by 13a and 13b as well as the elastic for the leg bands 19 and 20 may have alternate methods of construction, such as by folding over the material to form a sleeve, inserting the elastic within the sleeve, and fixing it within the sleeve by sewing it. The crotch 16 is sewn to the bottom edge 27 of rear panel 14 and the top edge 28 of front panel 15. Rectangular reinforcement strips 5 and 6 are sewn onto edges 22 and 23, respectively of rear panel 14. Snaps 1 and 2 are then attached to reinforcement panel 5, and snaps 3 and 4 are attached to reinforcement panel 6. In the same manner, rectangular reinforcement strips 11 and 12 are sewn onto edges 24 and 25, respectively of front panel 15, and receiving snaps 7 and 8 are attached to reinforcement panel 11, and receiving snaps 9 and 10 are attached to reinforcement panel 12.

As best seen in FIG. 1, snap underwear 21 fits like traditional underwear; that is, with a waistband 13 that holds the underwear 21 around the waist and hips, and with leg bands 19 and 20 that fit loosely around the legs. It should be noted that this loose fit is in contrast to the tight or snug fit found in diapers and training pants or panties. This loose fit provides an advantage to the child wearing the underwear because of the increased comfort and psychological impact of the underwear having a different feel than the training pants or panties from which the child has just graduated. Moreover, for those with mobility or agility problems, the use of otherwise conventional underwear but for the ability to easily remove and replace the undergarment provides increased comfort and psychological self-esteem because they know they are not wearing diapers or the like.

FIG. 2 shows that snap underwear 21 has a front panel 15 and a rear panel 14, each being somewhat rectangular in shape. They are joined together at the crotch 16, which is also somewhat rectangular in shape. Each of the panels 14 and 15 has an interior surface and an exterior surface and two opposing side edges. Along the length of each side edge 22, 23 of the rear panel 14, reinforcement strips 5, 6 have been sewn. Within reinforcement strip 5, snaps 1 and 2 are attached in a lengthwise orientation facing the interior of the underwear. Within reinforcement strip 6, snaps 3 and 4 are also attached in a lengthwise orientation facing the interior of the underwear. The front panel 15 is similarly configured, with reinforcement strips 11 and 12 sewn along the length of each side edge 24, 25. Within reinforcement strip 11, receiving snaps 7, 8 are attached facing the interior of the underwear. Likewise, within reinforcement strip 12, receiving snaps 9, 10 are attached facing the interior of the underwear.

Snaps 1 and 2 are spaced close enough in relation to each other to result in a smooth seam when connected to receiving snaps 7 and 8. Likewise, snaps 3 and 4 are spaced closely enough together to result in a smooth seam when connected to receiving snaps 9 and 10.

Although the drawings show a snap connecting means, a separate embodiment of the invention could use other connecting means, such as hook and loop or button and buttonhole, or cloth strips that extend from the front panel to the rear panel. Also, the number of snaps required in the reinforcement strips 5, 6, 11, 12 may increase depending on the size of the underwear 21 and the corresponding increase or decrease in the length of the side edges 22, 23, 24, 25. More snaps might be required so as to adequately hold the side edges 22, 23, 24, 25 together, and to prevent inadvertent unfastening of the snaps.

The snap underwear 21 is designed to be conveniently removed and replaced in those infrequent instances when the child soils the underwear. As best seen in FIG. 1, in practice, removing the underwear requires the child or parent (or other adult) to pull down clothing articles such as pants, shorts, or tights to the child's knees, and then unsnap snaps 1, 2, 3, 4 on rear panel 14 from their respective receiving snaps 7, 8, 9, 10 on front panel 15. The snap underwear 21 may then be pulled from between the child's legs toward the front of the child or through the child's legs toward the rear of the child. Once removed, the snap underwear 21 may be washed and re-used.

Replacing the snap underwear 21 requires the child or parent (or other adult) to pull down clothing articles such as pants, shorts, or tights to the child's knees, place the underwear 21 behind the child's buttocks with the interior of the pant against the child's buttocks. The front panel 15 is then pulled between the legs toward the front of the child. The side edge 23 of the rear panel 14 is then brought from in back of the child around the child's side. Side edge 23 is then lined up with side 25 of the front panel 15 with reinforcement strip 6 overlapping the outside of reinforcement strip 12, such that snap 3 can be secured to receiving snap 10 to partially form the waistband 13. In the same manner, side edges 22 and 24 are lined up with reinforcement strip 5 overlapping the outside of reinforcement strip 11, such that snap 1 can be secured to receiving snap 8 to complete the waistband 13 enclosure. Next, rear panel 14 snaps 2 and 4 are secured to front panel 15 receiving snaps 7 and 9, respectively to complete the leg bands 19 and 20 that form the leg holes 17 and 18. The resulting snap underwear 21 resembles the drawing in FIG. 1.

The snap underwear 21 can also be removed and replaced in the conventional way; that is, by removing any articles of clothing and footwear that might preclude removal or replacement, pulling the underwear 21 down the legs, over the knees, and off the feet, or performing those actions in reverse order.

It is important to note several aspects of this invention. First, the leg bands 19 and 20 formed are not tight so as to restrict the child's movement or be uncomfortable. This differs from diapers in which the wearer's leg pass through an opening, often termed the casing, which forms a tight fit around the leg. This is because there is no need to make the leg bands tight or bunch up to retain excrement and the like. Also, the rear panel 14 reinforcement strips 5, 6 with snaps 1, 2, 3, 4 and front panel 15 reinforcement strips 11, 12 with receiving snaps 7, 8, 9, 10 when joined are neither uncomfortable against the child's skin, nor are they bulky, which allows them to easily fit under typical children's clothes.

Finally, the material selected for the undergarment is of a type and thickness that is similar to conventional underwear. This is because the undergarment is not designed to be absorbent like diapers or training underwear. Diapers use materials, such as an acrylate polymer, that are highly absorbent. For example, the absorbance may be measured using demand wettability or gravimetric absorbance. These tests evaluate what is commonly referred to as the Absorbance Under Load (AUL). The AUL is defined as the amount of 0.9% saline solution absorbed by the polymers while being subjected to pressure equivalent to 21,000 dynes, or about 0.30 lb/sq in (0.021 kg/sq cm). This test simulates the effect of a baby sitting on a wet diaper. It is reported that if the diaper has an absorbance of at least 24 ml/g after one hour, the quality is considered acceptable. Other reports state that some diaper materials, such as sodium polyacrylate, can absorb 50 gallons of water for one pound of the sodium polyacrylate. In contrast, the absorbance of cloth underwear is significantly less because the cloth underwear does not use a material, e.g., that is designed to absorb water. The material selected for the undergarment is a material that has an absorbance significantly less than that of the material used in diapers, for example, less that 24 ml/gram; more particular, less than 20 ml/gram and even more particularly, less than 15 ml/gram and most particularly, less than 10 ml/gram. The thickest part is likely to be only the crotch region of the undergarment, just like conventional undergarment is thicker in the crotch region.

Conventional cloth diapers also will provide a greater absorbance than the undergarment based on using more material than the undergarment. Cloth diapers are recommended to be able to offer almost double the absorbency capacity of a baby's average wetting. One report states that an average newborn to 6 month old baby wets approximately 60-110 ml. during the day or at night; an average 6 month to 1 year old baby wets approximately 110-140 ml. during the day and 143-182 ml. at night; and an average toddler wets approximately 130-160 ml. during the day and 195-240 ml. at night. One method described for determining the absorbency of a diaper is as follows: (1) weigh one cloth diaper dry and write down the weight; (2) soak the diaper in at least 1000 ml. (4 cups) of water for one minute, remove the sopping wet diaper and hang to drip-dry for 15 minutes (do not wring our the fluid); (3) weigh the wet diaper. The absorbency capacity is calculated by subtracting the dry weight from the wet weight. For example, if the wet weight is 250 grams and the dry weight is 150 grams, then the absorbency capacity of the diaper would be 100 ml. Because the weight of 1 ml. of water is 1 gram, one can calculate how much weight of water the cloth diaper can absorb. This measurement does not directly take into account the inherent absorbance of the diaper material but more relies upon the thickness of the diaper. This thickness, however, provides another measure of the difference between the undergarment of the invention and cloth diapers.

An alternative embodiment of the snap underwear 21 is for the front panel 15 reinforcement strips 5, 6 to overlap the rear panel 14 reinforcement strips 11, 12. In this embodiment, the location of the snaps 1, 2, 3, 4 and receiving snaps 7, 8, 9 10 would be switched with each other.

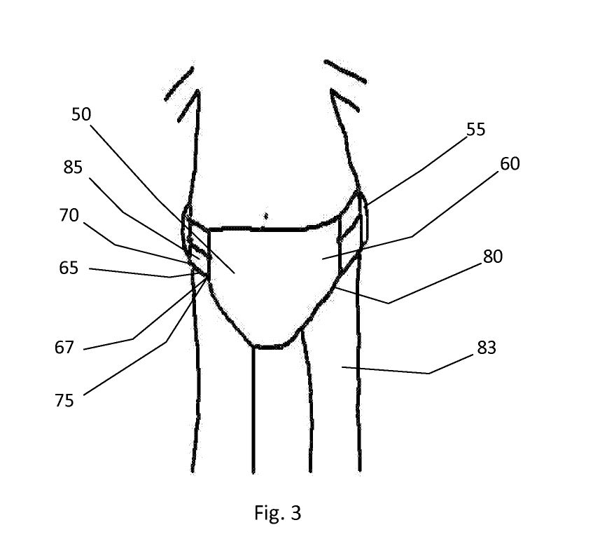

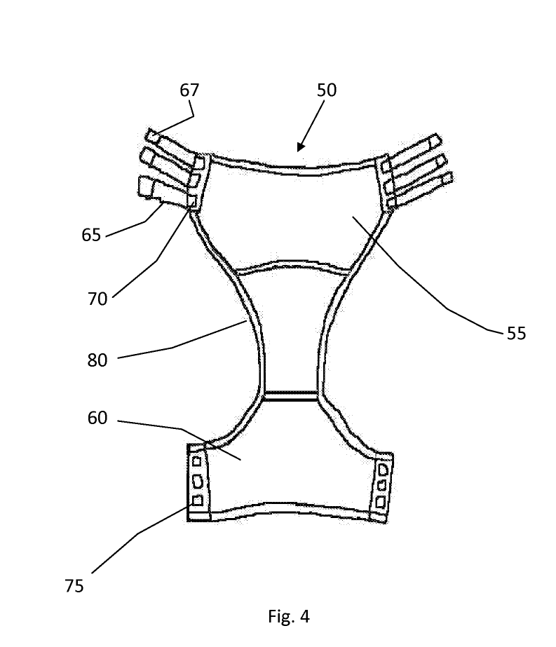

In alternative embodiments or along with the embodiments described above, the undergarments may have designs or logos embroidered, stitched, printed, etc. on them. The undergarments may be configured to allow for charms to be hung. As one example, the connecting means, such as the snap, may include a loop to which a charm can be hung. Similarly, referring to FIGS. 3 and 4, and as noted above, in an alternative embodiment an undergarment 50 can include one or more cloth strips 65 that extend between the front panel 60 and the rear panel 55. The cloth strips 65 are fastened at an end 70 of the rear panel and have a first connecting means 67 that mates with a second connecting means 75 on the front panel 60. When the undergarment 50 is worn and the cloth strips connected, the garment forms leg openings 80 through which the wearer's legs 83 pass. The arrangement of the cloth strips 65 and panels 55, 60 are such that the panels do not meet thereby forming an open space 85 between the panels 55, 60. The cloth strips 65 do not need to be of any particular material, design or configuration. Similarly, the connecting means 67, 75 do not need to be of any particular design and can be hooks and loops, snaps, fasteners, etc., or any of the other conventional fastening equipment described herein and/or conventionally used.

As described above, the undergarments herein can be used for children as well as for people of limited mobility and agility, such as the elderly, hospitalized, handicapped, maternity, infirm, etc. The undergarment also can be used by people in general, not just children or those of limited mobility and agility. The undergarment provides methods for personnel at hospital, nursing home, etc. to easily change underwear. In addition, patients themselves can more easily replace undergarments or get dressed by themselves with more ease.

While several particular forms of the invention have been illustrated and described, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited, except as by the appended claims.

What is claimed is:

1. An easily removable fabric undergarment comprising:
a front panel having an upper edge and two side panels extending from opposite sides of the front panel, each side panel terminating in a side edge that includes a first connecting means;
a rear panel having an upper edge and two side panels extending from opposite sides of the front panel, each side panel terminating in a side edge that includes a second connecting means; and
a crotch panel formed as part of one or both of the front panel and the rear panel, the crotch panel having a fabric of one or more layers,
wherein the fabric of the undergarment is of minimal absorbance and each of the first connecting means is configured to mate with the opposite second connecting means to form a closed waist loop for encircling the waist of the person wearing the undergarment formed by the front panel, the rear panel, and the side panels, and a pair of closed leg loops for encircling the legs of the person wearing the undergarment, the leg loops and waist loop each being configured to have a repeatable and consistent diameter and the leg loops being free of bunching up around the legs when worn.

2. The undergarment of claim 1, wherein the side panels extending from the front panel extend further than the side panels extending from the rear panel.

3. The undergarment of claim 1, wherein the side panels extending from the rear panel extend further than the side panels extending from the front panel.

4. The undergarment of claim 1, wherein one of the side panels extends from the front panel further than the mating panel that extends from the rear panel and the other side flap extends further from the rear panel than the mating panel that extends from the front panel.

5. The undergarment of claim 1, wherein the connecting means comprises one of a button or button hole.

6. The undergarment of claim 1, wherein the connecting means comprises a snap and a receiving snap.

7. The undergarment of claim 1, wherein the leg loops fit loosely around the legs when the person wears the undergarment.

8. The undergarment of claim 1, wherein the side edges comprise a reinforced fabric.

9. The undergarment of claim 1, wherein the side edges comprise a reinforced fabric on an inner side of the side edge and an outer side of the side edge.

10. The undergarment of claim 1, wherein the fabric has an absorbance less than a diaper or training underwear.

11. The undergarment of claim 1, wherein the fabric consists of a single layer.

12. The undergarment of claim 1, wherein the fabric consists of at least one layer.

13. A method of changing a fabric undergarment on an individual without the need to remove outer garments from the legs of the individual, the method comprising:
providing the undergarment to an individual, the undergarment comprising a front panel and a rear panel, the front panel having an upper edge and two side panels extending from opposite sides of the front panel, each side panel terminating in a side edge that includes a first connecting means, and the rear panel having an upper edge and two side panels extending from opposite sides of the front panel, each side panel terminating in a side edge that includes a second connecting means, wherein each of the first connecting means is configured to mate with the opposite second connecting means to form a closed waist loop for encircling the waist of the child wearing the undergarment formed by the front panel, the rear panel, and the side panels, and a pair of closed leg loops for encircling the legs of the child wearing the undergarment, the leg loops and waist loop each being configured to have a repeatable and consistent diameter; and
removing the undergarment from the individual without removing the outer garments in their entirety from the legs of the individual wearing the outer garment by unfastening the connecting means on the side panels and pulling the undergarment between the legs.

14. The method of claim 13, further comprising placing the undergarment on the individual without removing the outer garments in their entirety from the legs of the individual wearing the outer garment by pulling the undergarment between the legs and fastening the connecting means on the side panels.

15. The method of claim 13, wherein the side panels extending from the front panel extend further than the side panels extending from the rear panel.

16. The method of claim 13, wherein the side panels extending from the rear panel extend further than the side panels extending from the front panel.

17. The method of claim 13, wherein the leg loops fit loosely around the legs when the child wears the undergarment.

18. The method of claim 13, wherein the connecting means comprises one of a button or buttonhole, and a snap and a receiving snap.

19. The method of claim 13, wherein the side edges comprise a reinforced fabric.

20. The method of claim 13, wherein the fabric has a minimal absorbance.
